(19) United States
(12) Patent Application Publication
(10) Pub. No.: US 2005/0108048 A1
(43) Pub. Date: May 19, 2005

(54) Methods and system for managing mouse colonies

(75) Inventors: Robert Taft, South West Harbor, ME (US); Michael Wiles, Somesville, ME (US)

Correspondence Address: FISH & NEAVE IP GROUP;ROPES & GRAY LLP, ONE INTERNATIONAL PLACE, BOSTON, MA, 02110-2624, US

(73) Assignee: The Jackson Laboratory, Bar Harbor, ME (US)

(21) Appl. No.: 10/772,993

(22) Filed: Feb. 5, 2004

Related U.S. Application Data
(60) Provisional application No. 60/523,282, filed on Nov. 18, 2003.

(51) Int. Cl. G06F 17/60

(52) U.S. Cl. 705/2

(57) ABSTRACT

The present invention provides methods and systems for efficient and cost-effective provision of mouse colony management services to customers. Described herein is a novel integrated approach to provide a plurality of mouse colony management services by utilizing a common core process shared by the plurality of services.

### CROSS-REFERENCE TO RELATED APPLICATIONS

The present application claims the benefit of U.S. Provisional Application No. 60/523,282, filed Nov. 18, 2003, the specification of which is hereby incorporated by reference in its entirety.

### BACKGROUND OF THE INVENTION

The mouse is an excellent model system to study the genetics of complex human diseases. Random and selective techniques for altering the mouse genome provide powerful tools for biomedical research. Strains carrying specific mutations provide experimental systems for understanding gene function, for studying defects involved in specific human genetic diseases, for preclinical testing of therapeutic agents, and for developing new therapeutic interventions.

The number of mouse strains used for research has increased greatly due to the advent of transgenic and targeted mutation technology. This places increasing demand for efficient and effective ways to manage mouse colonies. One major bottleneck in the mouse colony management is the maintenance of thousands of new mutant mouse lines and the rapid distribution of these lines throughout the scientific community for researchers to use to investigate gene function and diseases. See Marschall and Angelis, Cryopreservation of mouse spermatozoa, Trends in Genetics 15: 128-131 (1999). Maintaining a large number of strains is critical to ensure continuity and advancement in biomedical research. However, maintenance of colonies of live mice is expensive. One solution is to maintain mouse strains through cryopreservation of mouse embryos. Presently, however, services to cryopreserve mouse embryos are slow and costly.

In addition to the task of maintaining mouse strains, other mouse colony management tasks also need to improve in efficiency, especially in light of greatly increased number of mouse strains. These mouse colony management tasks include, for example, rapid colony expansion, pathogen-free rederivation of a strain, rescue of a strain when there is only one mouse remains, and production of large waves of synchronized progeny with a specified genotype.

There is thus a pressing need for efficient, economic and reliable methods for managing mouse colonies.

### SUMMARY OF THE INVENTION

The present invention provides methods and systems for efficient and cost-effective provision of mouse colony management services to customers. Described herein is a novel integrated approach to provide a plurality of mouse colony management services by utilizing a common core process shared by the plurality of services. The provision of mouse colony management services can be streamlined by having multiple services share such a common core process. As a result, the overall process efficiency is improved, and the cost is consequently reduced. Moreover, the creative use of assisted reproductive technology to produce embryos improves efficiency of managing mouse colonies at the individual task level, as illustrated in more detail for each service in the following sections.

Managing mouse colonies involves controlled propagation of mice to give rise to desired live progeny and/or embryos, for various purposes. Mouse colony management thus relies, to a large extent, on breeding mice, which has traditionally been achieved through conventional mating. Relying on mating to produce live progeny and embryos has several limitations. First, the timing of mating is hard to control, making it difficult to produce desired live progeny and/or embryos on demand. Second, mating requires caging male and female mice together for a period of time to facilitate mating. This in turn requires expensive shelf space. Third, a given male can only mate with a limited number of females within a given time period, limiting the number of progenies and embryos that can be produced from a given male. Fourth, if a conventional breeding colony becomes infected, the infection will propagate, through mating, from generation to generation.

The methods of the present invention circumvent these biological constraints of mating through the innovative use of assisted reproductive technology. The term “assisted reproductive technology” (ART), as used herein, refers to any technology that manipulates the mouse reproductive process. The use of ART allows for control of at least two variables of mating: the timing of the reproduction and the number of embryos and/or live progeny produced. Thus, the use of ART gives one temporal control and numeric control of the mouse reproductive process. ART includes, for example, in vitro fertilization (IVF), artificial insemination, oocyte in vitro maturation, embryo transfer, intracytoplasmic sperm injection, cloning, in vitro culture of fertilized oocytes, embryo splitting, ovarian transplant, and regeneration of live progeny from ES cells, including regeneration of sperm and/or eggs from ES cells.

The use of ART gives one better and more effective control of the mouse reproductive process. This is exemplified in at least four aspects. First, assisted reproductive technologies, such as IVF and artificial insemination, allow one to control the precise timing of the union between a female gamete and a male gamete. Such precise control of timing can be crucial at times. For example, the erratic nature of mating makes it prohibitively expensive to produce a large number of synchronized progeny with desired genotypes. The term “synchronized progeny” refers to live progeny that are born within a short time frame from each other. Such short time frame may be, for example, 12 hours, 24 hours, 48 hours, 72 hours or 7 days. Due to the difficulty of timing the mating, producing a large number of synchronized progeny will require caging a much larger number of mating pairs together, a process likely to be prohibitively expensive. The use of IVF, an ART technology, circumvents this problem. Because IVF permits precise control of the timing of the union between a female gamete and a male gamete, fewer mice are needed to produce a large number of synchronized progeny than would be needed if conventional mating were used. As a result, the cost for providing synchronized progeny is reduced.

Second, using assisted reproductive technology to produce embryos does not require caging female and male mice together for a period of time to facilitate mating, thus reducing the need for expensive shelf space and at the same time reducing the time required to produce a given number of embryos.

Third, assisted reproductive technologies can enhance the reproductive process. For example, the use of IVF circumvents the biological limitation on how many times a given male can mate. By using IVF, the number of embryos/progeny that can be produced from a given male is limited only by the number of superovulated females available. ART can also be used to effectively rescue a strain that is on the verge of extinction. For example, if the only mouse left is a male, IVF can be used to produce offspring from the male. If the only mouse left is a female, oocyte in vitro maturation can be used to recover oocytes from a female mouse that is infertile, anestrus or reproductively senescent. The in vitro matured oocytes can then be used in IVF, thus maximizing the chance of producing offspring from the only female mouse.

Fourth, ART can be used to avoid the contact of infected parent mice with their progenies, thus preventing an infection to pass from one generation to the next. As a result, pathogen-free progeny can be produced from pathogen-ridden parent mice and infections can thus be eliminated from the strain.

By reducing the number of mice required, reducing the time involved, as well as enhancing the mouse reproductive process, the methods of the present invention permit one to produce embryos/live progeny more efficiently. It allows for provision of mouse colony management services that are both on demand and scalable. As a result, the use of ART as a core process for providing mouse colony management can directly translate into economic benefit of reduced costs.

Accordingly, one aspect of the present invention provides methods and systems for providing a plurality of mouse colony management services to customers by utilizing a core process and a service-specific process. The core process is shared among multiple mouse colony management services provided and the service-specific process is a process tailored for a particular service or a sub-group of services. The core process comprises producing embryos from donor female and donor male mice using an assisted reproductive technology. Either the donor male or the donor female mice, or both, may carry a desired trait, and may be provided by a customer or a third party. The plurality of mouse colony management services include generation and cryopreservation of mouse embryos, rapid expansion of a mouse colony, rapid production of synchronized progenies site-to-site transfer of a mouse strain, pathogen-free rederivation of a mouse strain, mouse embryo supply, supply of live mice recovered from cryopreserved embryos, rapid production of congenic strains with desired trait(s) in a desired inbred strain background, rapid production of congenic strains with desired phenotype(s) in a desired inbred strain background, and any combination of the above.

The present invention also provides kits for distributing cyropreserved embryos. The kits contain at least one cryopreserved embryo, a washing reagent for washing the cryoprotective solution off the embryo, and instructions for recovery of the cryopreserved embryo. To facilitate successful recovery of the cryopreserved embryos, at least one test cryopreserved embryo may be included in the kit for the customer to use in practicing the recovery procedure. In addition, recovery instructions may be included in the kits in a manner such that the instructions must be removed in order to provide access to the embryos. In one embodiment, the recovery instructions are integrated into the opening mechanism of the container containing the cryopreserved embryos such that the instructions must be removed in order to open the container. For example, the instructions are part of a shrink wrapper for the container. For another example, the instructions are affixed to (e.g., taped) the lid of the container. For a further example, the container is sealed with a strip, which may pass through a hole created in the instructions to keep it attached to the container. The container may be a single container, or may be a layered container with an inner container and an outer container.

Practice of these methods will allow one to provide multiple mouse colony management services more efficiently and economically than can be provided using the presently available methods.

### BRIEF DESCRIPTION OF THE DRAWINGS

Embodiments of the invention are illustrated by way of example, and not by way of limitation, in the figures of the accompanying drawings.

FIG. 1 is a flow diagram that presents an overview of the methods of the present invention.

FIG. 2 is a flow diagram that depicts an approach for generating and providing mouse embryos.

FIG. 3 is a flow diagram that depicts a method for rapid colony expansion.

FIG. 4 is a flow diagram that depicts a method for rapid production of synchronized progeny.

FIG. 5 is a flow diagram that depicts an approach for generating and cryopreserving mouse embryos.

FIG. 6 is a flow diagram that depicts an approach for generating, cryopreserving mouse embryos and recovery of cryopreserved embryos.

FIG. 7 is a flow diagram that depicts an approach for site to site transfer of embryos.

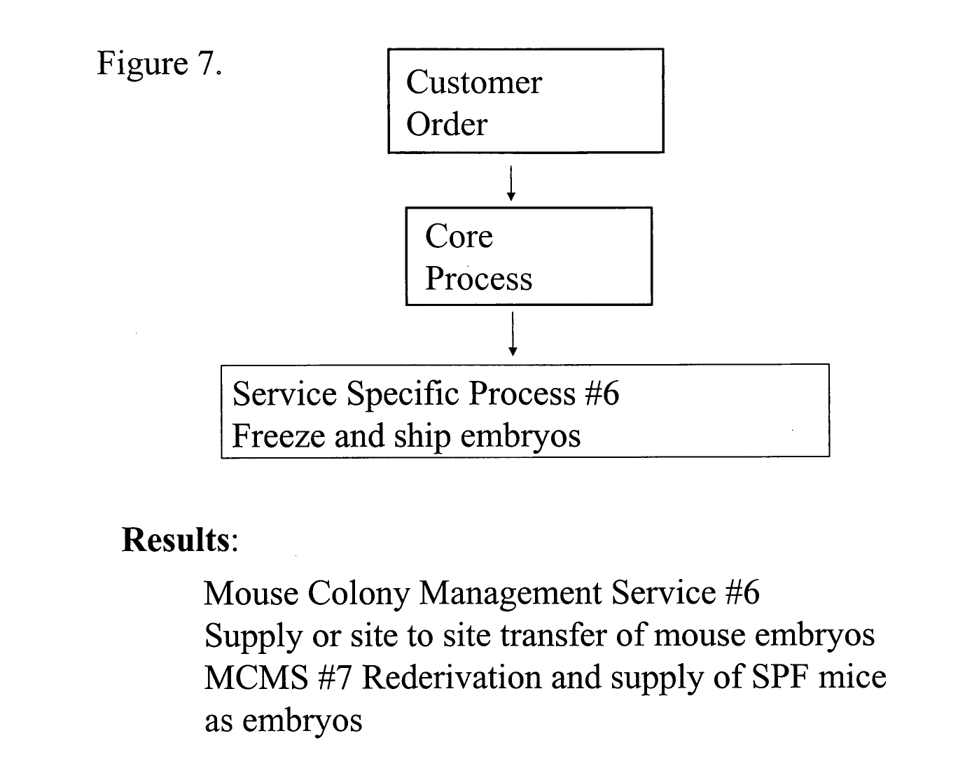

FIG. 8 is a flow diagram that depicts an approach for rapid production of congenic strains.

FIG. 9 is a flow diagram that depicts an approach for rapid production of congenic strains for mapping of a desired phenotype.

FIG. 10 is a schematic comparison of colony management by IVF and by conventional mating.

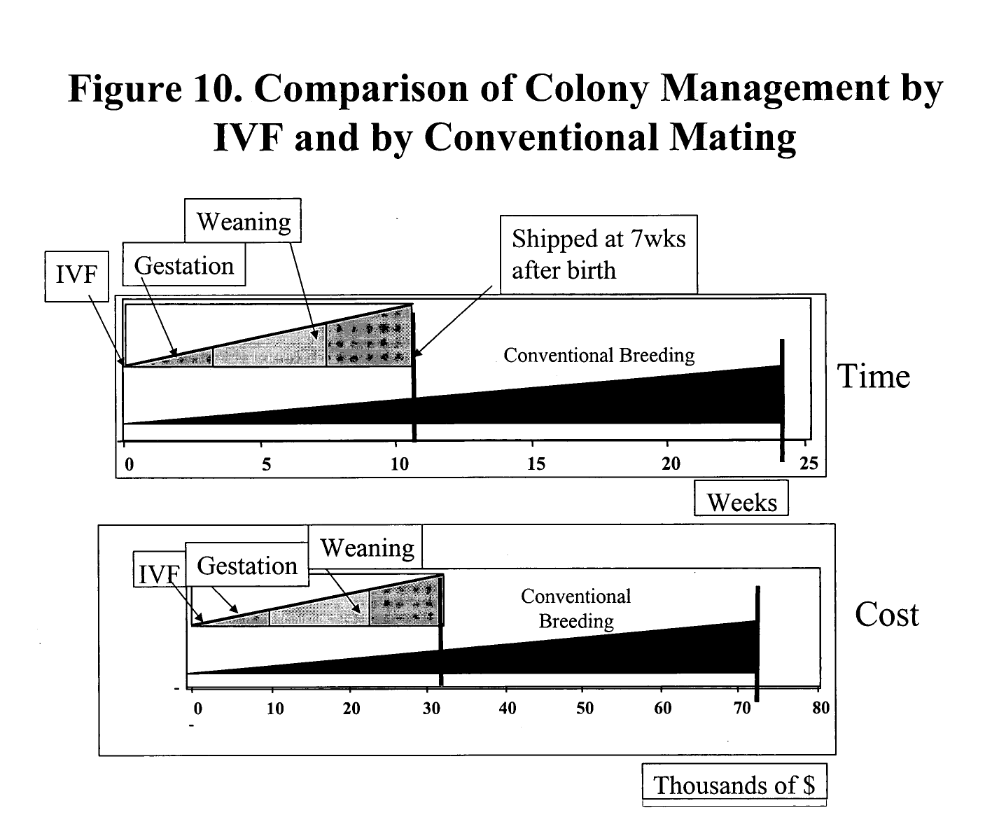

### DETAILED DESCRIPTION OF THE INVENTION

I. Methods

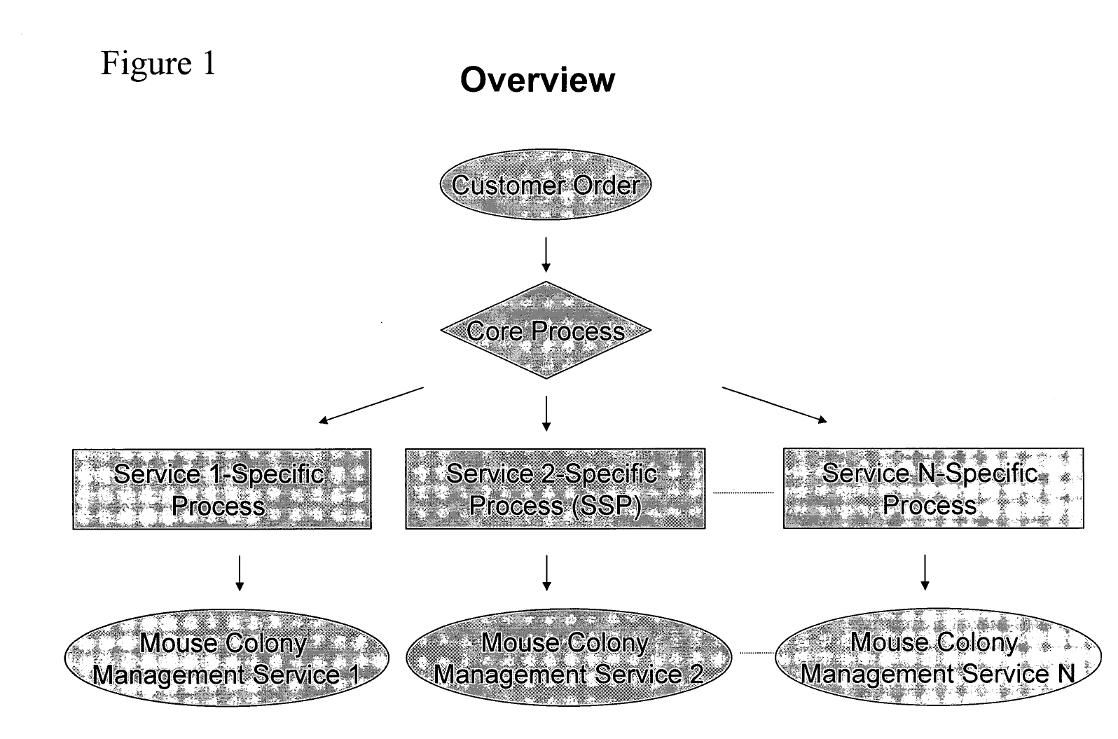

FIG. 1 is a flowchart depicting an overview of the methods of the present invention. Briefly, the methods start with receiving at least one order from a customer specifying the type(s) of service(s) desired. In one embodiment, the customer also provides appropriate materials to generate desired embryos. In another embodiment, a third party or the service provider provides appropriate materials to generate desired embryos. A core process is then carried out to produce desired embryos, followed with a service-specific process.

The term “mouse colony management services”, as used herein, refers to processes that help one manage mouse colonies. Such mouse colony management services include, for example, generation and provision of mouse embryos, generation and cryopreservation of mouse embryos, storage and recovery of cryopreserved mouse embryos, rapid colony expansion, rapid production of synchronized progeny, site-to-site transfer of mice, pathogen-free rederivation of mice, rapid production of congenic strains with desired trait(s) in a desired inbred strain background, rapid production of congenic strains with desired phenotype(s) in a desired inbred strain background, and any combination of the above.

Depending on the services desired, the appropriate materials provided by a customer include one or a combination of the following: a male mouse with desired trait(s), a female mouse with desired trait(s), embryos from mice with a desired trait, sperm, oocytes and/or ovaries from mice with desired trait(s). The term “trait”, as used herein, includes both genetic traits and phenotypic traits (phenotypes). A desired genetic trait may or may not have a readily discernible phenotype. For example, a mouse with a desired genetic trait may be a mouse in which an endogenous gene is disrupted, but no discernible phenotype is associated with the gene disruption. For another example, a mouse with a desired genetic trait may be a mouse with a transgene inserted into its genome. Again, such transgenic mouse may not display any discernible difference in phenotype, as compared to a wild type mouse which does not include a transgene. A desired genetic trait may be generated by transgenic or knockout technologies. Mice generated by transgenic or knockout technologies may be further crossed to produce mice with multiple desired genetic traits.

Alternatively, embryos may be generated using a combination of materials provided by a customer and materials provided by the service provider. For example, a customer may provide a male mouse with a desired mutation in a gene, and the service provider may provide a female mouse that is wild type for the gene.

The core process may be any process that is shared among a plurality of mouse colony management services. In one embodiment, the core process is producing embryos from a given pair of male and female mice using an assisted reproductive technology. ART includes, for example, in vitro fertilization (IVF), artificial insemination, oocyte in vitro maturation, embryo transfer, intracytoplasmic sperm injection, cloning, in vitro culture of fertilized oocytes embryo splitting, ovarian transplant, and regeneration of live progeny from ES cells, including regeneration of sperm and/or eggs from ES cells. By producing embryos using ART instead of by mating, the methods of the present invention can be readily scaled up or down, in response to customers' needs.

The “service-specific process” refers to a process that is tailored for a particular service or a sub-group of services. The process may comprise one step, or may comprise multiple steps. One or more steps of a service-specific process may be shared among several services provided, further improving efficiency and reducing cost for providing the plurality of mouse colony management services.

In one embodiment, the core process is producing embryo by in vitro fertilization, which comprises: (1) superovulating a donor female mouse, (2) obtaining oocytes from the superovulated donor female mouse, (3) obtaining sperm from a donor male mouse having a desired trait, (4) fertilizing in vitro oocytes obtained in (b) with sperm obtained in (c), thereby producing fertilized oocytes, (5) culturing fertilized oocytes produced in (d) in culture media under conditions appropriate for development of fertilized oocytes into embryos, whereby embryos are produced, and (6) harvesting embryos from the culture media.

In vitro fertilization (IVF) is well known in the art. See, for example, Hogan et al., Manipulating the Mouse Embryos, A Laboratory Manual, 2^{nd} Ed. Page 146-147 (1994). Embryos may be harvested at different stages. The female may be superovulated before oocytes are collected for IVF. See, for example, Hogan et al., Manipulating the Mouse Embryos, A Laboratory Manual, 2^{nd} Ed. page 130-133.

The steps of the IVF core process can be modified to accommodate customers' individual needs. For example, a customer may desire to obtain a large number of embryos from a single female donor mouse. This may be achieved by several approaches.

In one approach, the production of a large number of embryos can be achieved by in vitro maturation of oocytes as an alternative to collecting mature oocytes for IVF from the donor female. Thus, in one embodiment, immature oocytes may be obtained from the donor female and cultured in vitro under conditions that result in the maturation of these oocytes, a technique known as “in vitro maturation”. In mammals, only a small fraction of immature oocytes develop into mature oocytes, and the rest degenerate and die. By isolating immature oocytes from a donor female and allowing them to mature in vitro, one can obtain many more oocytes suitable for IVF from the donor female than can be obtained by collecting mature oocytes directly from the female. Mammalian oocytes are known to undergo maturation in vitro and give rise to normal healthy offspring when embryos are transferred to an appropriate uterus (Schroeder and Eppig 1984 Dev. Biol. 102:493; Sirar et al. 1988, Biol.Reprod. 39:546). In vitro maturation technique is well known in the art. See, for example, Chiu et al., Effects of Myo-inositol on the in-vitro Maturation and Subsequent Development of Mouse Oocytes, Human Reprod. 18: 408-416 (2003) and O'Brien et al., A Revised Protocol for In Vitro Development of Mouse Oocytes from Primordial Follicles Dramatically Improves Their Developmental Competence, Biol. Reprod. 68: 1682-1686 (2003).

In an alternative embodiment, oocytes may be collected from a host female into whom a section of ovaries from the donor female had previously been implanted. This is achieved by harvesting ovaries from the donor female, sub-dividing the ovaries into sections, implanting each section into an ovarisectomized host female, and collecting oocytes from each of the host females after sufficient time to allow the transplanted ovary section to develop into a functional ovary. This approach results in more oocytes obtained from the donor female.

In a further embodiment, the step of obtaining oocytes in the IVF core process comprises repetitive superovulation of the donor female and oocyte collection.

In an another embodiment, intracytoplasmic sperm injection (ICSI) may be used to improve fertilization rate in IVF. The ICSI procedure is suitable for poor quality sperm, but may be used for any sperm. The ICSI procedure involves removal of the cumulus cells surrounding oocytes and injection of the sperm head into the oocytes, ordinarily through a glass pipette. See Kimura and Yanagimachi, 1995.

The present invention also encompasses other variations of the IVF core process. For example, instead of obtaining sperm from a donor male mouse supplied by a customer, a service provider may offer customers the option of collecting sperm from a desired donor male mouse at their sites, and shipping the sperm to the service provider. Likewise, a service provider may offer customers the option of collecting oocytes from a desired donor female mouse at their sites, and shipping the oocytes to the service provider for IVF. The term “service provider”, as used herein, refers to an entity which provides mouse colony management services.

In another embodiment, the core process is producing embryos by artificial insemination. Artificial insemination is a process of fertilizing female animals by manual introduction or application of sperm. In such a procedure, male animals are not required at the time of insemination, as sperm is obtained from them previously. See Wolfe, 1967, and Sato and Kumura, 2002. When breeding is achieved by artificial insemination, embryos may be obtained by flushing the oviduct or uterus of the female after artificial insemination. See Hogan et al., 2003.

In a further embodiment, the core process is producing embryos from embryonic stem cells. Embryonic stem cells (ES cell) are pluripotent cells isolated from blastocyst stage embryos. Embryonic stem cells may be cultured in vitro with or without feeder cells. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335, both of which are incorporated herein by reference in their entireties. The term “embryonic stem cell”, or “ES cell”, as used herein, includes ES cell lines.

The ES cell may be cryopreserved and stored. Preferably, the ES cells are cyropreserved at an early passage, such as, for example, within 3-20 passages. ES cells may be cryopreserved using method known to those skilled in the art. See, for example, Hogan et al., 2003.

Cryopreserved ES cells can be thawed and used to produce embryos to regenerate live progeny. In one embodiment, ES cells of the invention may be introduced to a morula or into a blastocyst stage embryo and can contribute to the development of the animal. The resulting offspring are typically chimeras, with portions of the animals developed from either the host embryo or the contributing ES cells. The germline of the Chimera could be derivatives of both the host and the contributing ES cells. The resulting male and female chimeras are regenerate live progeny. By the use of coat color and/or other genetic markers, one can select offspring derived completely from the ES cell.

In an alternative embodiment, ES cells are introduced into a tetraploid host embryo. Such ES cells may successfully fully colonize the mouse embryo proper, while the extraembryonic tissue is supplied by the tetraploid host. This will give rise to offspring that are completely derived from the ES cells. Appropriate monitoring of the progeny using coat color and/or other indicators of genetic markers can distinguish any possible host-embryo-derived offspring from those derived from the ES cells. One can thus select offspring derived completely from ES cells.

Such techniques are known in the art. See, for example, Hogan et al., 2003. Derivation of completely cell culture-derived mice from early-passage embryonic stem cells, Proc Natl Acad Sci U S A. 90, 8424-8428. See also
http://www.mshri.on.ca/nagy/diploid/diploid.htm, and
http://www.mshri.on.ca/nagy/Tetraploid/tetra.htm

A donor mouse may be supplied by the service provider, by a customer, or by a third party supplier. In one embodiment, a donor male mouse is supplied by a customer, while donor female mice are supplied by the service provider. Alternatively, a customer may supply a donor female mouse, and the service provider may supply a donor male mouse. In a further embodiment, a customer may supply both male and female donor mice.

Types of mouse strain of the present invention include all research mouse model strains, including inbred strains, congenic strains, recombinant-inbred strains, hybrids, outbreds, and induced mutant strains.

Mouse inbred strains of the present invention include, but are not limited to, 129S1, 129T2, 129X1, 129P3, 129P1, A, AKR, BALB/c, C3H, C57BL/10, C57BL/6, C57BLKS, C57BR/cd, C57L, CBA, DBA/1, DBA/2, FVB, MRL, NOD, SJL, SWR, NOR, NZB, NZW, RBF, BTBR T+tf, BUB, I, LP, NON, P, PL, RIIS, SM, C58, ALR, ALS, BPH, BPL, BPN, DDY, EL, KK, LG, MA, NH, NZM2410, NZO, RF, SB, SEA, SI, SOD1, YBR, and all substrains of each of these strains. The term “substrains”, as used herein, refers to colonies within the same strain that are genetically different from each other.

The induced mutant strains include mouse strains generated by transgenic, knockout, or siRNA technologies. An induced mutant strain has either a transgene inserted in its genome, or an endogenous gene harboring a mutation, such as, for example, a deletion, an insertion, a frame shift or a point mutation.

Such induced mutant strains often have mixed genetic backgrounds. A strain with mixed genetic background is a strain in which the genetic background is derived from more than one type of strain. For example, breeding a male BL/6 with a female 129T2 will give rise to a mouse with a mix of BL/6 and 129T2 background.

Mice or embryos provided to a customer can be homozygous or heterozygous for a given genotype, depending on the customer's order. To generate a homozygous mouse or embryo, a customer may provide both a male and a female mouse with the desired genetic trait. Progeny generated from these mice will be homozygous. Alternatively, a customer may provide only a male mouse with the desired genetic trait. This may be necessitated when the female mice with the desired genetic trait are infertile. When a customer provides only a male mouse with the desired genetic trait and requests homozygous progeny, the F1 generation progeny will be crossed to generate F2 generation. About a quarter of the F2 generation thus generated will be mice or embryos homozygous for the desired genetic trait. These F2 generation mice or embryos can be shipped to the customer.

In one embodiment, the female mice used in assisted reproductive technology, such as IVF, are of known genetic background. Services provided may be priced based on the genetic background of the female mice used. In this case, price quotes for services can be given without a test run first to assess the efficiency of embryo production for the strain. In a further embodiment, the pricing is based on genetic background, but is further modified to include considerations for certain mutant strains with reduced fertility in the males or females.

Embryos used in the methods of the present invention may be any stage embryos that can be successfully cryopreserved and recovered to produce live animals. Thus, an embryo used in the methods of the present invention may be, for example, a one-cell embryo (a zygote), a two-cell embryo, a four-cell embryo, an eight-cell embryo, a morula or a blastocyst. A morula is a spherical mass of cells resulting from a cleaved embryo, and a blastocyst is an embryo having a blastocoel. In one embodiment, the embryos used in the methods of the present invention are 2-cell stage embryos.

In one embodiment, provision of a plurality of mouse colony management services is coordinated through scheduling and data management services.

Different types of mouse colony management services are described in detail in the following sections. All of the above description is applicable to each service. For illustrative purposes, IVF is used as a core process in these sections. It is evident to one skilled in the art that other ART may be used as a core process to provide mouse colony management services as well. Furthermore, it is possible to use one assisted reproductive technology as a core process for a sub-group of services, while using another assisted reproductive technology as a core process for another sub-group of services.

Generation and Provision of Mouse Embryos

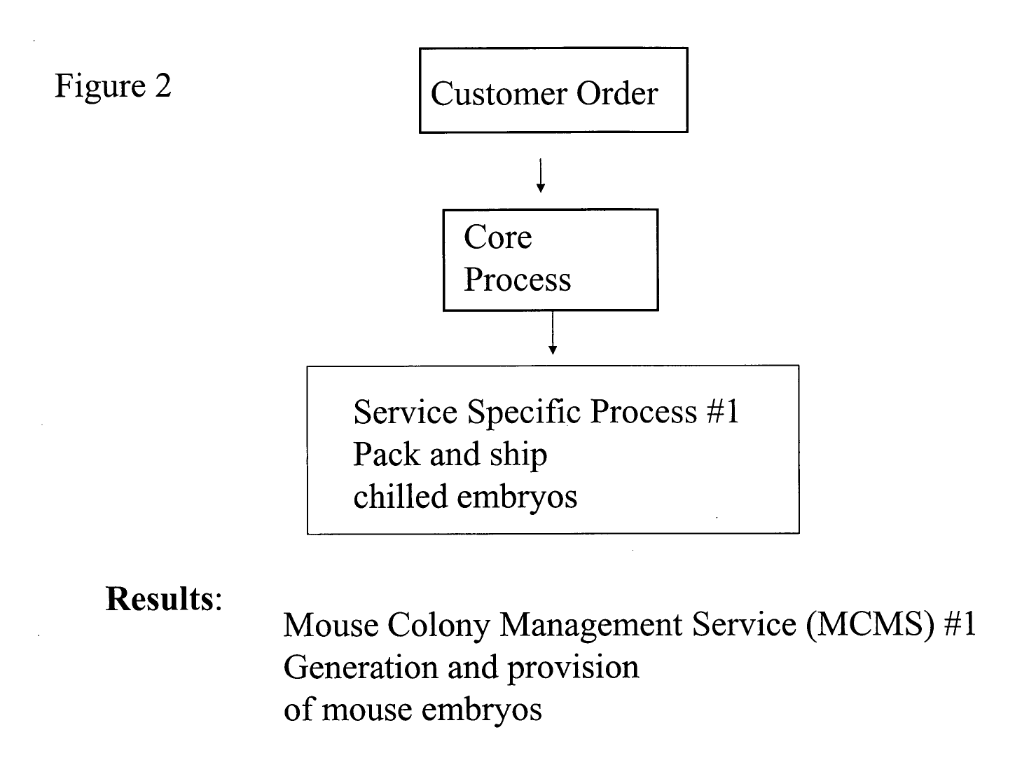

The methods of the present invention may be used to generate and provide mouse embryos to customers. As illustrated in FIG. 2, after receiving an order specifying the services desired and appropriate materials from a customer, a core process of in vitro fertilization is carried out to produce desired embryos. Depending on the services desired, the materials provided by a customer include one or a combination of the following: a male mouse with desired trait(s), a female mouse with desired trait(s), sperm and/or oocytes from mice with desired trait(s). Embryos may be generated using only materials provided by a customer. Alternatively, embryos may be generated using a combination of materials provided by a customer and materials provided by the service provider. For example, a customer may provide a male mouse with a desired trait(s), and the service provider may provide a female mouse with wild type background. After the embryos are produced, they are packed and shipped to the customer.

Rapid Colony Expansion, Rederivation of Strains, Rescue of Strains

Faster methods of mouse colony expansion are needed for the establishment of a new mouse line, or for speeding up and enlarging backcross, outcross or intercross projects. Currently, these projects are slow, expensive and space-demanding. See Marschall and Angelis, Cryopreservation of mouse spermatozoa, Trends in Genetics 15: 128-131 (1999).

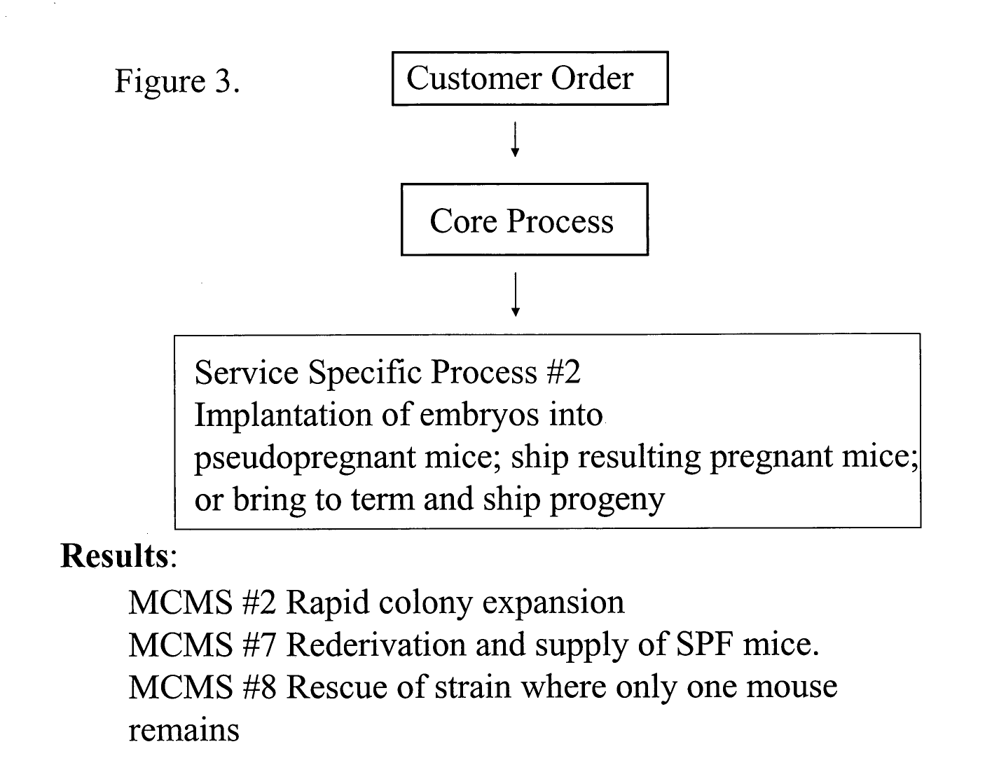

FIG. 3 depicts an approach for rapid expansion of a mouse colony using the methods of the present invention. Upon receiving an order specifying the types of services desired and appropriate materials as described above, a core process of in vitro fertilization is first carried out to produce a desired number of embryos. After embryos are produced, a service-specific process is carried out, which comprises implanting the embryos produced into at least one pseudopregnant female mouse. The resulting female mouse or mice can be supplied to the customer directly. Alternatively, the resulting female mouse or mice may be maintained under conditions suitable to bring them to term. The progeny thus produced are shipped to the customer. The process can be scaled up or down, depending on the needs of customers.

The rapid colony expansion can be used to expand from an existing colony, or to expand from small stocks such as a single transgenic mouse or a single knockout mouse.

The same process can be used for pathogen-free rederivation of a mouse colony. The process can also be used to rescue a strain where only a small number of mouse remains, including, for example, only one mouse remains. Where there is only one male mouse left in a strain, IVF can be performed using this male as the donor male and a wild type female as the donor female. The only male mouse left may have reduced fertility (for example, it may be old or decrepit). Alternatively, the last male mouse left may have been recently deceased. In these situations, ICSI (a form of IVF, as described above) may be used to produce embryos. Embryos produced can be implanted into pseudopregnant female mice, which are then brought to term to produce live progeny. These will be further bred to produce homozygous progeny if desired.

Rapid Production of Desired Number of Synchronized Progeny

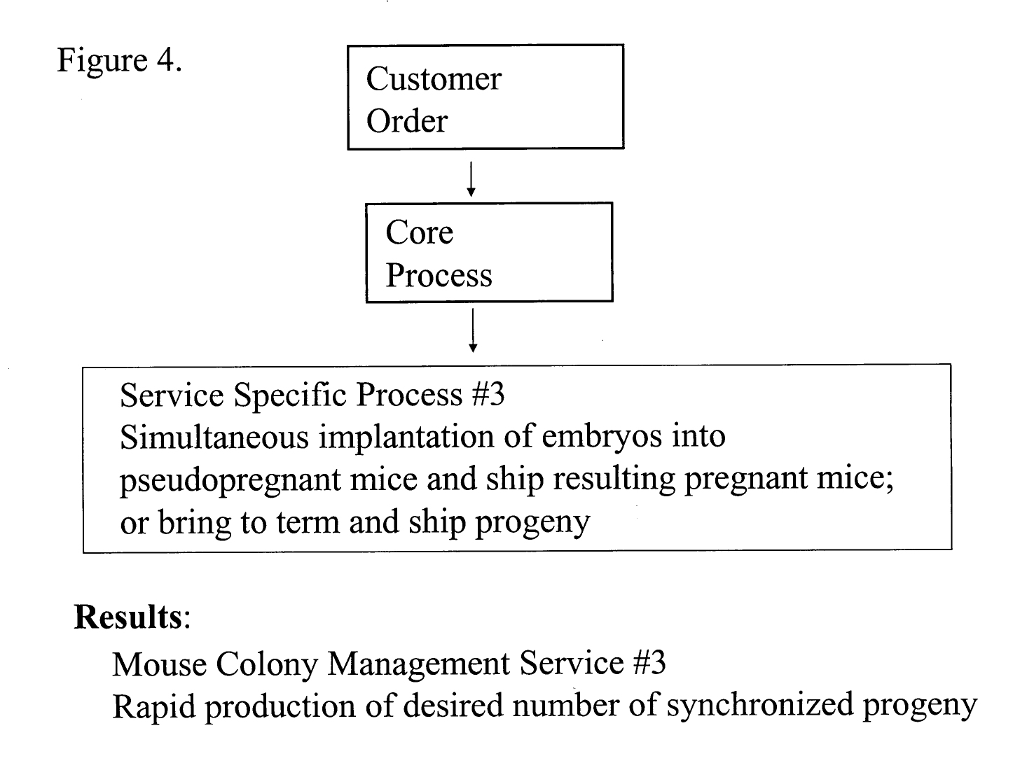

FIG. 4 depicts an approach for rapid production of a desired number of synchronized progeny with the same genotype. The term “synchronized progeny” refers to progeny that are born at substantially the same time, such as, for example, within 24 hours of each other. It may be desirable to produce a large number of mice with the same genotype within a short time frame from each other. Such short time frame may be, for example, 12 hours, 24 hours, 48 hours, 72 hours or 7 days. This is difficult to achieve with conventional mating, partly due to the difficulty of coordinating the timing of mating to ensure the simultaneous birth of a large number of progeny. The problem is compounded by the resources needed to cage a large number of mice. The use of IVF facilitates such production economically and efficiently.

According to the present invention, upon receiving an order and appropriate materials from a customer, a core process is carried out to produce desired embryos. A service-specific process is then carried out, which comprises simultaneously implanting the embryos into an appropriate number of pseudopregnant female mice. These female mice may be shipped to the customer. Alternatively, progeny are born and shipped to the customer.

A customer can order the production of a desired number of synchronized mice with a certain genotype from a pre-existing cryopreserved stock. This pre-existing stock may be previously created for the same customer, or a part of common stock created by the service provider.

Generation, Cryopreservation of Mouse Embryos and Storage of Cryopreserved Embryos; Recovery of Mouse Embryos

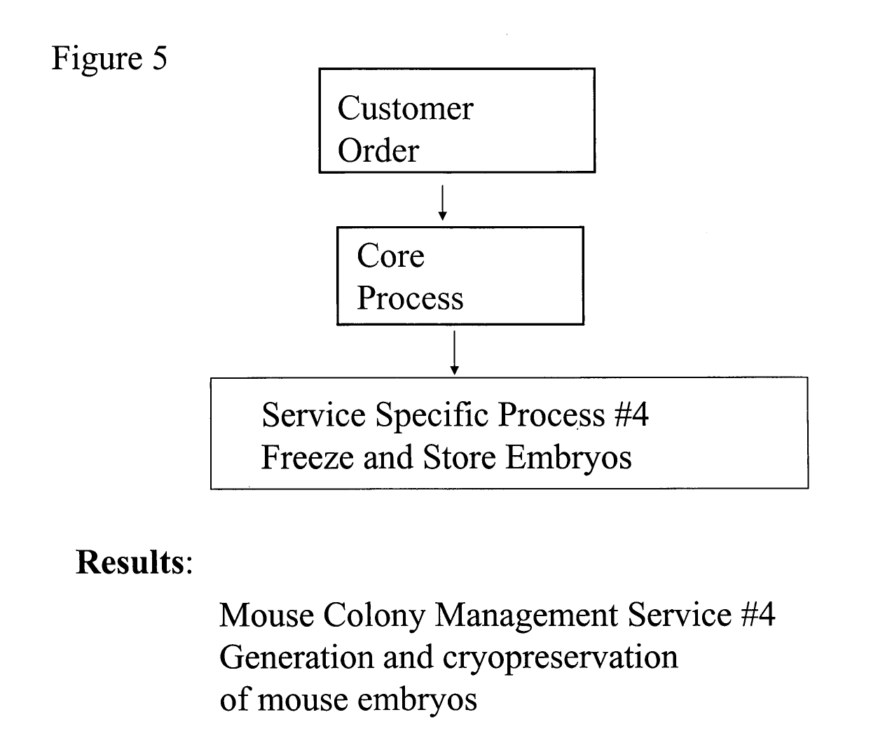

FIG. 5 depicts an approach for generating and cyropreserving mouse embryos using the methods of the present invention. A desirable alternative to maintaining live mice is cryopreservation of mouse embryos carrying a desired trait(s). Mice with a desired trait(s) can be recovered any time a need for live mice arises. Upon receiving an order specifying the services desired and appropriate materials from a customer, a core process of in vitro fertilization is carried out to produce desired embryos. Depending on the services desired, the materials from a customer include, for example, male and/or female mouse with desired traits, sperm and/or oocytes from mice with desired traits. These embryos are then cyropreserved and stored at the location of the service provider. The service provider may maintain a customer account to track the cryopreserved stock that are maintained at the service provider for the customer. Account reports may be generated and sent to customers at regular intervals.

The term “cryopreserved” as used herein refers to being frozen. The embryos used in the methods are frozen at temperatures generally lower than 0° C. For examples, −80° C. can be used for short term storage, and −196° C. or lower can be used for long term storage. Embryos used in the methods of the present invention can be cryopreserved for an indefinite length of time. Methods and tools for cryopreservation are well-known to those skilled in the art. See, e.g., U.S. Pat. No. 5,160,312, entitled “Cryopreservation Process for Direct Transfer of Embryos”, Glenister and Hall, “Cryopreservation and rederivation of embryos and gametes”, in Mouse Genetics & Transgenics: A Practical Approach, 2^{nd} Edition (I Jackson & C Abbott, eds.) Oxford Univ. Press, Oxford, pp. 27-29.

The cryopreserved embryos may be stored by methods known to those skilled in the art. In one embodiment, cryopreserved embryos are stored in sterile plastic insemination straws. Alternatively, cryopreserved embryos are stored in plastic vials or glass ampoules.

After embryos are cryopreserved and stored, a customer may desire to recover them at some point. Three options are available for customers. First, the cryopreserved embryos can be packed and shipped directly to the customer. Alternatively, the cryopreserved embryos are thawed and implanted into pseudopregnant female mice, and the resulting pregnant mice are shipped to the customer. In the third option, the cryopreserved embryos are thawed and implanted into pregnant female mice, and the resulting pregnant mice are maintained under conditions suitable for production of live offspring. The offspring thus produced are then shipped to the customer. A customer can order cryopreserved embryos from his/her own stock, from another customer's stock with consent, or from a common stock maintained by the service provider.

Provision of Cryopreserved Embryos; Site to Site Transfer of Mice as Embryos; Rederivation and Supply of Specific-Pathogen-Free (SPF) Mice as Embryos

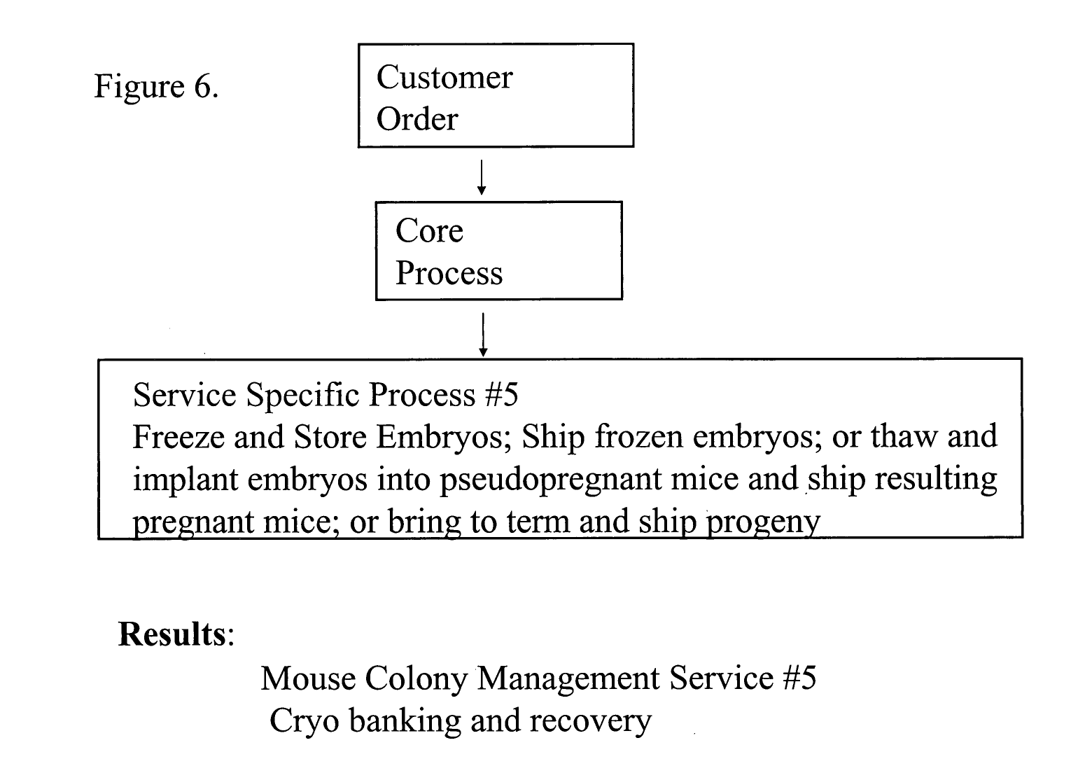

Building on the generation and cryopreservation of mouse embryo services, FIG. 6 depicts an approach for generating and cryopreserving mouse embryos and provision of cryopreserved embryos. Upon receiving a customer order, a core process of IVF is carried out to produce embryos. The embryos are then cryopreserved and shipped to customers.

This process can be used to facilitate site-to-site transfer of mice. Shipping live mice is costly and slow, due to the fact that live mice would have to go through quarantine upon arrival at a new site. In contrast, shipping frozen embryos is more cost-effective and faster, without the hassle of handling live animals. In one embodiment, a customer supplies at least one mouse with desired trait(s). A core process of IVF is carried out to produce embryos. The embryos are then cryopreserved and shipped to a third party, as requested by the customer.

This process is also useful for rederivation and supply of SPF mice as embryos. Mice of interest are sometimes afflicted with viral or bacterial infection, hindering research and/or transfer of these to desired sites. The process described in this section can be used to produce pathogen-free embryos from afflicted mice. In one embodiment, a customer supplies at least one afflicted mouse with desired trait. A core process of IVF is carried out to produce embryos that are pathogen-free. The embryos are then cryopreserved and shipped to either the customer or a third party, as requested by the customer.

Production of Congenic Strains with Desired Genetic Traits

Genetically-engineered mice carrying targeted mutations or transgenes are often initially generated and characterized on a mixture of genetic background. Furthermore, new alleles may appear on genetic backgrounds that are inappropriate for future investigation. Congenic strain development allows for the movement of genetic loci from one strain background to another. For example, for a knockout allele generated in a strain with mixed C57BL/6 and 129 background, successive backcrossing between the donor knockout strain and a recipient inbred strain C57BL/6 will eventually produce a congenic strain with the knockout allele in C57BL/6 background. As used herein, the starting strain background will be termed the “donor background”, whereas the recipient strain background will be termed the “target background”.

Congenic strains offer several advantages over analysis of genes or transgenes on an undefined or mixed genetic background. First, it provides a defined experimental model for characterization and uses. Second, it reduces experimental variability, thereby reducing the total number of mice needed per experiment.

Typically, the congenic animals are identified from progeny in the range of 8-12 generations. Alternatively, a procedure referred to as “speed congenics” or “marker-assisted breeding” can be used. See, for example, Production of Congenic Strains Using Marker-Assisted (“Speed”) Technologies, JAX Communication, No. 6 (November 2001), Whittaker et al., Genet. Res., 66(3):255-265, 1995; and Darvasi, Nat. Genet., 18(1):19-24, 1998. In this method, genomic markers are used to genotype the progeny to select for progeny in each backcross generation that have lost the maximum number of donor background alleles. Such genomic markers include, for example, DNA microsatellite markers. For example, the presence of microsatellites or simple sequence length polymorphisms (SSLPs), composed of mono-, di-, tri-, or tetrameric sequences repeated multiple times in a tandem array, can be assessed by amplification of the region surrounding a microsatellite or SSLP using the polymerase chain reaction (PCR). For an example of DNA microsatellite markers, see The Mouse Genome Database at www.informatics.jax.org. In this method, less breeding is required. As a result, the generation time to create a congenic strain can be significantly reduced. In addition to shortening the time required, the total number of mice needed in the congenic strain development process is also reduced.

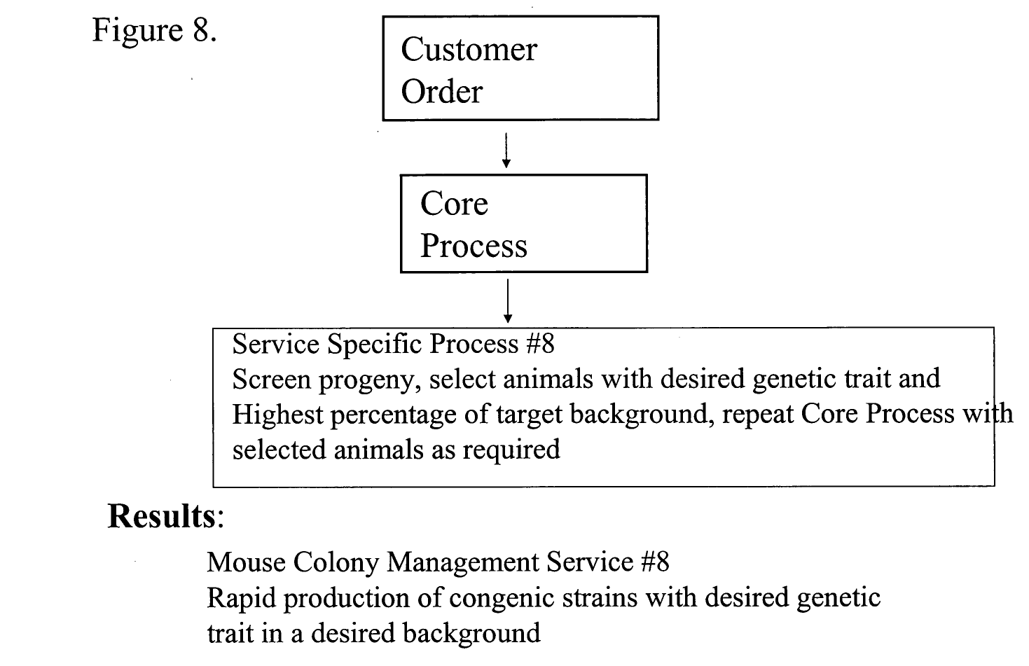

The present invention provides methods of further reducing the time required to develop a congenic strains by utilizing the core process of an assisted reproductive technology, such as, for example IVF. In one embodiment, upon receiving an order from a customer, a core process of IVF is carried out to produce embryos. After the embryos are produced, a service-specific process is carried out, which comprises: (1) implanting the embryos into at least one pseudopregnant recipient mouse, (2) maintaining the recipient(s) under conditions which result in production of live progeny, (3) among live progeny produced, selecting progeny with both the desired allele of interest and the highest percentage of target background, and (4) repeating the core process and the service-specific process as necessary to produce a congenic mouse with the allele of interest in a desired target background. The steps are depicted in FIG. 8.

In certain instances, step (3) in the above-described service-specific process may be modified. For example, when a live progeny produced after a round of breeding has a marker(s) in the vicinity of the allele of interest, that progeny may be selected for the next round of breeding, even though this progeny may not be the one with the highest percentage of target background. In all subsequent rounds of breeding, the progeny with both the marker and the highest percentage of target background is selected. Having a marker close to the allele of interest facilitates future crosses to select for the allele of interest. Thus, in an alternative embodiment, the service-specific process comprises the above-described steps, except that whenever a live progeny produced has a marker(s) in the vicinity of the gene of interest, step (3) becomes: among live progeny produced, selecting progeny with both the desired marker (associated with desired allele of interest) and the highest percentage of target background.

Production of Congenic Strains with Desired Phenotypes

The process of producing congenic strains can also be used to map gene(s) associated with a desired phenotype. This may be desirable for naturally-arising mutations, or for mutations generated by a random mutagenesis program. In this scenario, a desired phenotype is found on one genetic background, and this starting strain is termed donor strain. To identify the gene(s) responsible for the phenotype, the donor strain is successively backcrossed with a recipient host strain. The resulting progeny are screened to select for the desired phenotype while monitoring genomic markers. Co-segregation of genomic marker(s) with the desired phenotype indicate that the gene responsible for the desired phenotype resides in the area where the genomic marker(s) is linked to. The procedure of “speed congenics” or “marker-assisted breeding”, as described above, can be used for this process.

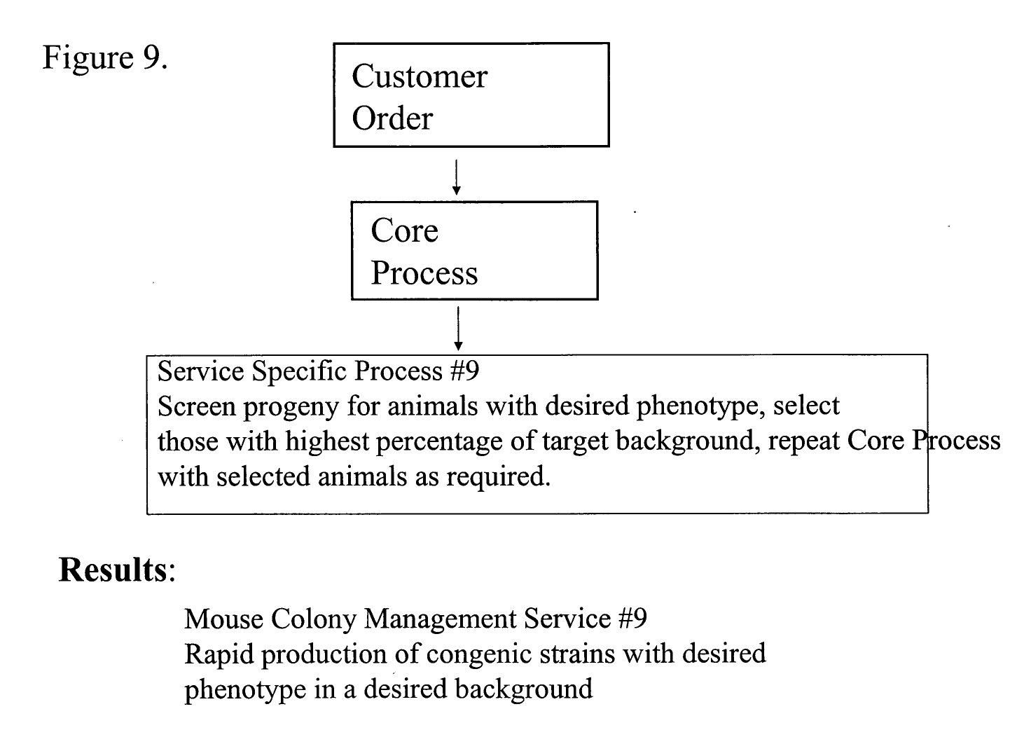

The present invention provides methods to reduce the time required to develop congenic strains by utilizing the core process of an assisted reproductive technology, such as, for example IVF. In one embodiment, upon receiving an order from a customer, a core process of IVF is carried out to produce embryos. After the embryos are produced, a service-specific process is carried out, which comprises: (1) implanting the embryos into at least one pseudopregnant recipient mouse to produce live progeny, (2) among live progeny produced, selecting progeny with both the desired phenotype and the highest percentage of the target background, and (3) repeating the core process and the service-specific process as necessary to produce a congenic mouse with desired phenotype(s) in a desired background. This process is useful to narrow the area of search for the gene(s) associated with the desired phenotype. The steps are depicted in FIG. 9.

II. System

Another aspect of the present invention relates to systems used to provide a plurality of mouse colony management services. The system comprises at least four of the following modules: a customer service module, a scheduling and data management module, a live animal module, a surgery and assisted reproductive technology module; a cryopreservation module, a packing and shipping module and an education and training module.

The customer service module comprises means to interface with customers. Interfacing with customers includes handling contracts, reviewing and responding to technical and business inquiries, dealing with legal issues, providing quotes for available services, sending out quality control reports and reports about the level of cryopreserved stock and maintaining customer contact database.

The scheduling and data management module comprises means to set up and coordinate the schedule of all modules and to manage data generated. This module is used to manage customer embryo accounts and generate customer reports providing information on customer embryo accounts. Such information may include, for example, account number, customer name, transaction history and the number of cryopreserved embryos left in the account. In one embodiment, the scheduling and data management module comprises a customer tracking system, which gives customers the option to track the progress of their order. Once a customer places an order, he or she is given a tracking number, which can be used to obtain status reports on their order on an appropriate web page.

The live animal module comprises a barrier space for incoming animals, a barrier space for superovulated females, pseudopregnant females, and a barrier space for breeding and shipping.

The surgery and assisted reproductive technology module comprises means for performing surgery and carrying out assisted reproductive technologies. In one embodiment, this module is a surgery and in vitro fertilization module.

The cryopreservation module comprises means to freeze embryos and other appropriate biological materials, means for freezer space management and frozen stock back up management.

The packing and shipping module comprises means for coordinating the import and export of mice, embryos, DNA, tissue and other biological samples. For the purposes of bio-security, separate facilities for importing and exporting streams of animals may be included.

The education and training module comprises means for providing customer education materials, customer training courses and internal training and education.

To carry out the methods of the present invention, at least four of the modules are used to provide a mouse colony management service. Many of these modules are used and shared to provide the plurality of mouse colony management services. For example, in one embodiment, to provide a desired service to a customer, a service provider may utilize the customer service module to receive and process an incoming order, and use the surgery and in vitro fertilization module for breeding and producing desired embryos/live animals. A service provider may also utilize the live animal module for any incoming animals, superovulated females, pseudopregnant females, and for breeding and shipping, and use the packing and shipping unit for sending out embryos and/or live animals to customers. Each of these modules, especially the surgery and IVF module, may be repetitively accessed and utilized to provide a desired service/products to customers.

III. Kits

Another aspect of the present invention relates to kits for distributing cyropreserved embryos. The kits comprise at least one cryopreserved embryo, a washing reagent for washing the cryoprotective solution off the embryo, and instructions for recovery of the cryopreserved embryo. Recovery of cryopreserved embryos can be technically difficult. To facilitate successful recovery by customers, at least one test cryopreserved embryo may be included in the kit. In one embodiment, at least one straw of test embryos is included in the kit. A customer can use the test cryopreserved embryo to practice the recovery procedure. After becoming familiar with the recovery procedure, the customer can then recover the cryopreserved embryo of interest. The inclusion of test embryo will likely improve the recovery success of valuable cryopreserved embryos. Another way of improving recovery success is to improve the chance a customer will read the recovery instructions accompanying the kit. Accordingly, in one embodiment, the recovery instructions are included in the kits in a manner such that the instructions must be removed to provide access to the embryos. In one embodiment, the recovery instructions are integrated into the opening mechanism of the container containing the cryopreserved embryo such that the instructions must be removed in order to open the container. For example, the instructions are part of a shrink wrapper for the container. For another example, the instructions are affixed to (e.g., taped on) the lid of the container containing the cryopreserved embryo. For a further example, the container is sealed with a strip, which may pass through a hole created in the instructions to keep it attached to the container. The container may be a single container, or may be a layered container with an inner container and an outer container.

### EXEMPLIFICATION

The invention now being generally described, it will be more readily understood by reference to the following examples, which are included merely for purposes of illustration of certain aspects and embodiments of the present invention, and are not intended to limit the invention.

### EXAMPLE

### Production of 220 Synchronized Mice that are Within 1 Week of Age

In this project, a customer wanted 220 animals heterozygous for the gene of interest, all within 1 week of age. The customer provided a single heterozygous male carrying the gene of interest.

To carry out this project, we superovulated 25 three week old B6/129F1 by injecting them with 2.5 IU of PMSG and then induced ovulation by injecting them with 5 IU of hCG˜46 hrs later. 14 hours after the injection of hCG oocytes were collected and incubated with sperm from a heterozygous male carrying the gene of interest for 4 hours to allow fertilization to occur. At the end of this incubation, the oocytes were moved to fresh media and incubated overnight. The following morning, the resulting embryos were collected, washed and then transferred into 100 pseudopregnant females. 3 weeks later, 640 pups were born. These animals were genotyped at 4 weeks of age and the approximately 300 animals of the correct genotype were shipped to the customer 3 weeks later when pups are of wean age. The entire process, from the time the customer order and materials were received to the time mice were delivered, took about 10 weeks. The cost for production by IVF for this project is about $30,000.

In contrast, to produce 220 mice heterozygous for the gene of interest within 1 week of age by conventional mating would take about 24-28 weeks by conservative estimate. See Handbook on genetically standardized mice, Fox and Witham, Fifth Edition August 1997, The Jackson Laboratory. Specifically, production by conventional mating can be carried out by the following steps with the assumptions that the male provide by the customer is fertile, 90% of females produce a litter, females become pregnant the first week, female heterozygous for the gene of interest and male heterozygous for the gene of interest (“het”) are both fertile and average litter size is 7.

Steps:

1. Mate the het male provided by the customer to 2 females of an F1
2. rotate the het male to 2 new females weekly for 6 weeks.
3. Genotype males only and save all het male offspring. (2×0.9×7/4=3 carrier males per week for 6 weeks=18 males)
4. Once all 18 males are old enough to breed, trio mate to F1 females. (36×0.9×7=226 pups) 113 are carriers.
5. Pair mate the same day 50 het females and 50 het males to F1. 100×0.9×7=630 pups, of which 300 will be carriers born within a week of each other.

Based on the above calculations, production by conventional mating to generate and deliver about 300 pups of wean age as desired by the customer will take about 24-28 weeks, doubling the production time. In addition, production by conventional mating will entail accumulating sufficient numbers of mice before the final production could begin. This process will require caging of mice and their maintenance, resulting in additional costs. By conservative estimates, the project costs will amount to over $70,000 for production by conventional mating.

In conclusion, compared to production by conventional mating, production by IVF for this project cuts both the production time and the production costs by more than half, and is thus a superior method. The comparison is schematically represented in FIG. 9.

The practice of the present invention will employ, unless otherwise indicated, conventional techniques of mouse genetics, developmental biology, cell biology, cell culture, molecular biology, transgenic biology, microbiology, recombinant DNA, and immunology, which are within the skill of the art. Such techniques are described in the literature. See, for example, Current Protocols in Cell Biology, ed. by Bonifacino, Dasso, Lippincott-Schwartz, Harford, and Yamada, John Wiley and Sons, Inc., New York, 1999; Manipulating the Mouse Embryos, A Laboratory Manual, 3^{rd} Ed., by Hogan et al., Cold Spring Harbor Laboratory Press, Cold Spring Harbor, N.Y., 2003; Gene Targeting: A Practical Approach, IRL Press at Oxford University Press, Oxford, 1993; and Gene Targeting Protocols, Human Press, Totowa, N.J., 2000. All patents, patent applications and references cited herein are incorporated in their entirety by reference.

One skilled in the art readily appreciates that the present invention is well adapted to carry out the objects and obtain the ends and advantages mentioned, as well as those inherent therein. The methods, systems and kits are representative of preferred embodiments, are exemplary, and are not intended as limitations on the scope of the invention. Modifications therein and other uses will occur to those skilled in the art. These modifications are encompassed within the spirit of the invention and are defined by the scope of the claims. It will be readily apparent to a person skilled in the art that varying substitutions and modifications may be made to the invention disclosed herein without departing from the scope and spirit of the invention.

1. A method for providing a plurality of mouse colony management services to customers, comprising:
(a) receiving an order from a customer that indicates the service desired;
(b) providing the plurality of services to the customer by utilizing a core process and optionally, a service-specific process, whereby the core process is shared by the plurality of services.

2. The method of claim 1, wherein the core process is producing embryos using an assisted reproductive technology.

3. The method of claim 2, wherein the assisted reproductive technology is producing embryos by in vitro fertilization, comprising:
(a) superovulating a donor female mouse;
(b) obtaining oocytes from the superovulating donor female mouse;
(c) obtaining sperm from a donor male mouse having a desired trait;
(d) fertilizing in vitro oocytes obtained in (b) with sperm obtained in (c), thereby producing fertilized oocytes;
(e) culturing fertilized oocytes produced in (d) in culture media under conditions appropriate for development of fertilized oocytes into embryos, whereby embryos are produced.

4. The method of claim 3, wherein the plurality of mouse colony management services is selected from: generation and cryopreservation of mouse embryos; rapid expansion of a mouse colony; rapid production of synchronized progeny, site-to-site transfer of a mouse strain; pathogen-free rederivation of a mouse strain; strain rescue; mouse embryos supply; supply of live mice recovered from cryopreserved embryos; rapid production of congenic strains with desired genetic trait(s) and rapid production of congenic strains with desired phenotypes.

5. The method of claim 4, wherein the donor male or donor female mouse having a desired trait is provided by a customer or a third party.

6. The method of claim 4, wherein both the donor male mouse and the donor female mouse are provided by a customer or a third party.

7. The method of any one of claims 3-6, wherein the donor female mouse has a pre-determined genetic background.

8. The method of claim 7, wherein the embryos are harvested at 2-cell stage.

9. The method of claim 8, wherein pricing of the plurality of mouse colony management services is based on the genetic background of the donor female mouse.

10. The method of claim 9, wherein the mouse colony management service is rapid expansion of a mouse colony, and the service-specific process comprises:
(a) transferring an appropriate number of embryos produced by the core process into an appropriate number of pseudopregnant female mice;
(b) shipping the resulting pregnant mice to customers.

11. The method of claim 9, wherein the mouse colony management service is rapid expansion of a mouse colony, and the service-specific process comprises:
(a) transferring appropriate number of embryos produced by the core process into an appropriate number of pseudopregnant female mice;
(b) maintaining the resulting pregnant mice under conditions suitable for production of live progeny, thereby producing live progeny.
(c) shipping the live progeny to a customer.

12. The method of claim 9, wherein the mouse colony management service is generation and provision of mouse embryos with a desired trait, and the service-specific process comprises: packing and shipping to customer the embryos produced by the core process.

13. The method of claim 9, wherein the mouse colony management service is generation and cryopreservation of mouse embryo with desired trait, and the service-specific process comprises:
(a) cyropreserving embryos produced by the core process;
(b) storing the cryopreserved embryos, thereby producing a cyropreserved embryo stock.

14. The method of claim 9, wherein the mouse colony management service is generation and cryopreservation of mouse embryos and provision of cryopreserved embryos, and the service-specific process comprises:
(a) cyropreserving the embryos produced by the core process;
(b) storing the cryopreserved embryos, thereby producing a cyropreserved embryo stock;
(c) in response to a customer order, supplying the customer with the desired cryopreserved embryos from the cryopreserved embryo stock of claim 13.

15. The method of claim 9, wherein the mouse colony management service is generation and cryopreservation of mouse embryos and provision of live mice recovered from cryopreserved embryos, and the service-specific process comprises:
(a) cyropreserving the embryos produced by the core process;
(b) storing the cryopreserved embryos, thereby producing a cyropreserved embryo stock;
(c) in response to a customer order, thawing cryopreserved embryos from the cryopreserved embryo stock of claim 13, thereby producing thawed embryos;
(d) transferring thawed embryos into pseudopregnant female mice;
(e) maintaining resulting pregnant mice under conditions suitable for production of live progeny;
(f) supplying live progeny to the customer.

16. The method of claim 9, wherein the mouse colony management service is rapid production of desired number of synchronized progeny, and the service-specific process comprises:
(a) simultaneously transferring an appropriate number of embryos produced by the core process into appropriate number of pseudopregnant female mice;
(b) maintaining the resulting pregnant mice under conditions suitable for production of live progeny, thereby producing an appropriate number of live progeny; and
(c) shipping live progeny to the customer.

17. The method of claim 9, wherein the mouse colony management service is rapid production of congenic strains with desired genetic trait(s), and the service-specific process comprises:
(a) implanting embryos produced by the core process into at least one pseudopregnant recipient mouse;
(b) maintaining the resulting pregnant mice under conditions suitable for production of live progeny;
(c) among live progeny produced, selecting progeny with both the desired allele of interest and the highest percentage of target background; and
(d) repeating the core process and the service-specific process as necessary to produce a congenic mouse with the allele of interest in a desired target background.

18. The method of claim 9, wherein the mouse colony management service is rapid production of congenic strains with desired phenotype(s), and the service-specific process comprises:
(a) implanting embryos produced by the core process into at least one pseudopregnant recipient mouse;
(b) maintaining the resulting pregnant mice under conditions suitable for production of live progeny;
(c) among live progeny produced, selecting progeny with both the desired phenotype and the highest percentage of target background; and
(d) repeating the core process and the service-specific process as necessary to produce a congenic mouse with desired phenotype(s) in a desired target background.

19. A method for providing a plurality of mouse colony management services to customers, comprising:
(a) receiving an order from a customer that indicates the service desired; and
(b) providing the plurality of services to the customer by utilizing an assisted reproductive technology.

20. The method of claim 19, wherein the assisted reproductive technology is in vitro fertilization, comprising:
(a) superovulating a donor female mouse;
(b) obtaining oocytes from the superovulating donor female mouse;
(c) obtaining sperm from a donor male mouse having a desired trait;
(d) fertilizing in vitro oocytes obtained in (b) with sperm obtained in (c), thereby producing fertilized oocytes;
(e) culturing fertilized oocytes produced in (d) in culture media under conditions appropriate for development of fertilized oocytes into embryos, whereby embryos are produced.

21. The method of claim 20, wherein the plurality of mouse colony management services is selected from: generation and cryopreservation of mouse embryos; rapid expansion of a mouse colony; rapid production of synchronized progeny, site-to-site transfer of a mouse strain; pathogen-free rederivation of a mouse strain; strain rescue; mouse embryos supply; supply of live mice recovered from cryopreserved embryos; rapid production of congenic strains with desired genetic trait(s) and rapid production of congenic strains with desired phenotypes.

22. The method of claim 21, wherein the donor male or donor female mouse having a desired trait is provided by a customer or a third party.

23. The method of claim 21, wherein both the donor male mouse and the donor female mouse are provided by a customer or a third party.

24. The method of any one of claims 19-23, wherein the donor female mouse has a pre-determined genetic background.

25. The method of claim 24, wherein the embryos are harvested at 2-cell stage.

26. The method of claim 25, wherein pricing of the plurality of mouse colony management services is based on the genetic background of the donor female mouse.

27. A system for providing a plurality of mouse colony management services to customers, said system comprising at least three of the following modules:
(a) a customer service module;
(b) a scheduling and data management module;
(c) a live animal module;
(d) a surgery and in vitro fertilization module;
(e) a cryopreservation module;
(f) a packing and shipping module; and
(g) an education and training module.
wherein the plurality of mouse management services are provided to customers by utilizing a combination of at least four of these modules.

28. The system of claim 27 wherein the system is computer implemented.

29. The system of claim 28 wherein the live animal module comprises:
(a) a barrier space for incoming animals;
(b) superovulated females;
(c) pseudopregnant females; and
(d) a barrier space for breeding and shipping.

30. A kit for distributing cryopreserved embryos, comprising:
(a) at least one cryopreserved embryo;
(b) a washing reagent for washing the cryoprotective solution off the embryo; and
(c) instructions for recovery of the cryopreserved embryo.

31. The kit of claim 30, wherein the kit further comprises a cryopreserved test embryo.

32. The kit of claim 31, wherein the instructions for recovery of the cryopreserved embryo is included in the kit in a manner such that the instructions must be removed to provide access to the embryo.
